(19) United States
(12) Patent Application Publication
(10) Pub. No.: US 2002/0151702 A1
(43) Pub. Date: Oct. 17, 2002

(54) Aggrecanase molecules

(75) Inventors: Lisa A. Racie, Acton, MA (US); Natalie C. Twine, Goffstown, NH (US); Michael J. Agostino, Andover, MA (US); Neil Wolfman, Dover, MA (US); Elisabeth A. Morris, Sherborn, MA (US)

Correspondence Address: FINNEGAN, HENDERSON, FARABOW,, GARRETT & DUNNER, L.L.P., 1300 I Street, N.W., Washington, DC, 20005, US

(73) Assignee: Genetics Institute, Inc., Cambridge, MA (US)

(21) Appl. No.: 09/978,979

(22) Filed: Oct. 16, 2001

Related U.S. Application Data
(60) Provisional application No. 60/241,469, filed on Oct. 18, 2000.

(51) Int. Cl. C07H 21/04

(52) U.S. Cl. 536/23.2

(57) ABSTRACT

Novel aggrecanase proteins and the nucleotides sequences encoding them as well as processes for producing them are disclosed. Methods for developing inhibitors of the aggrecanase enzymes and antibodies to the enzymes for treatment of conditions characterized by the degradation of aggrecan are also disclosed.

### RELATED APPLICATION

This application claims prior from copending provisional application serial No. 60/241,469 filed on Oct. 18, 2000.

The present invention relates to the discovery of nucleotide sequences encoding novel aggrecanase molecules, the aggrecanase proteins and processes for producing them. The invention further relates to the development of inhibitors of, as well as antibodies to the aggrecanase enzymes. These inhibitors and antibodies may be useful for the treatment of various aggrecanase-associated conditions including osteoarthritis.

### BACKGROUND OF THE INVENTION

Aggrecan is a major extracellular component of articular cartilage. It is a proteoglycan responsible for providing cartilage with its mechanical properties of compressibility and elasticity. The loss of aggrecan has been implicated in the degradation of articular cartilage in arthritic diseases. Osteoarthritis is a debilitating disease which affects at least 30 million Americans [MacLean et al. J Rheumatol 25:2213-8. (1998)]. Osteoarthritis can severely reduce quality of life due to degradation of articular cartilage and the resulting chronic pain. An early and important characteristic of the osteoarthritic process is loss of aggrecan from the extracellular matrix [Brandt, K D. and Mankin H J. Pathogenesis of Osteoarthritis, in Textbook of Rheumatology, WB Saunders Company, Philadelphia, Pa. pgs. 1355-1373. (1993)]. The large, sugar-containing portion of aggrecan is thereby lost from the extra-cellular matrix, resulting in deficiencies in the biomechanical characteristics of the cartilage.

A proteolytic activity termed “aggrecanase” is thought to be responsible for the cleavage of aggrecan thereby having a role in cartilage degradation associated with osteoarthritis and inflammatory joint disease. Work has been conducted to identify the enzyme responsible for the degradation of aggrecan in human osteoarthritic cartilage. Two enzymatic cleavage sites have been identified within the interglobular domain of aggrecan. One (Asn³⁴1-Phe³⁴²) is observed to be cleaved by several known metalloproteases [Flannery, C R et al. J Biol Chem 267:1008-14. 1992; Fosang, A J et al. Biochemical J. 304:347-351. (1994)]. The aggrecan fragment found in human synovial fluid, and generated by IL-1 induced cartilage aggrecan cleavage is at the Glu³⁷³-Ala3⁷⁴ bond [Sandy, J D, et al. J Clin Invest 69:1512-1516. (1992); Lohmander L S, et al. Arthritis Rheum 36: 1214-1222. (1993); Sandy J D et al. J Biol Chem. 266: 8683-8685. (1991)], indicating that none of the known enzymes are responsible for aggrecan cleavage in vivo.

Recently, identification of two enzymes, aggrecanase-1 (ADAMTS 4) and aggrecanase-2 (ADAMTS-11) within the “Disintegrin-like and Metalloprotease with Thrombospondin type 1 motif” (ADAM-TS) family have been identified which are synthesized by IL-1 stimulated cartilage and cleave aggrecan at the appropriate site [Tortorella M D, et al Science 284:1664-6. (1999); Abbaszade, I, et al. J Biol Chem 274: 23443-23450. (1999)]. It is possible that these enzymes could be synthesized by osteoarthritic human articular cartilage. It is also contemplated that there are other, related enzymes in the ADAM-TS family which are capable of cleaving aggrecan at the Glu³⁷³-Ala3⁷⁴ bond and could contribute to aggrecan cleavage in osteoarthritis.

### SUMMARY OF THE INVENTION

The present invention is directed to the identification of aggrecanase protein molecules capable of cleaving aggrecanase, the nucleotide sequences which encode the aggrecanase enzymes, and processes for the production of aggrecanases. These enzymes are contemplated to be characterized as having proteolytic aggrecanase activity. The invention further includes compositions comprising these enzymes as well as antibodies to these enzymes. In addition, the invention includes methods for developing inhibitors of aggrecanase which block the enzyme's proteolytic activity. These inhibitors and antibodies may be used in various assays and therapies for treatment of conditions characterized by the degradation of articular cartilage.

The nucleotide sequence of the aggrecanase molecule of the present invention is set forth FIG. 1. As described in Example 1 the first 780 base pairs is a partial sequence of aggrecanase of the invention followed by the sequence of Hsa011374 deposited in Genbank accession no. AJ011374. The invention further includes equivalent degenerative codon sequences of the sequence set forth in FIG. 1, as well as fragments thereof which exhibit aggrecanase activity.

The amino acid sequence of an isolated aggrecanase molecule is set forth in SEQ ID. No. 1. The nucleotide sequence for this sequence is set forth in SEQ ID No. 2 and its complement SEQ ID No. 3. SEQ ID No 4 sets forth the nucleotide sequence for Hsa 011374 while SEQ ID No. 5 sets forth the amino acid sequence encoded by nucleotides #619 through #1710 of SEQ ID No. 4. Representing amino acids #207 through #570 in the first translated frame of the Hsa 011374 sequence. Amino acids #1-#737 of SEQ ID No. 6 are encoded by Hsa011374 representing the second translational frame. The invention further includes fragments of the amino acid sequence which encode molecules exhibiting aggrecanase activity.

The human aggrecanase protein or a fragment thereof may be produced by culturing a cell transformed with a DNA sequence of FIG. 1 or a DNA sequence comprising the sequence of SEQ ID. Nos. 2 or 3 and recovering and purifying from the culture medium a protein characterized by the amino acid sequence set forth in SEQ ID No. 1 substantially free from other proteinaceous materials with which it is co-produced. For production in mammalian cells, the DNA sequence further comprises a DNA sequence encoding a suitable propeptide 5′ to and linked in frame to the nucleotide sequence encoding the aggrecanase enzyme.

The invention includes methods for obtaining the full length aggrecanase molecule, the DNA sequence obtained by this method and the protein encoded thereby. The method for isolation of the full length sequence involves utilizing the aggrecanase sequence set forth in FIG. 1 or the sequences set forth in SEQ ID Nos. 2 and 3 to design probes for screening using standard procedures known to those skilled in the art.

It is expected that other species have DNA sequences homologous to human aggrecanase enzyme. The invention, therefore, includes methods for obtaining the DNA sequences encoding other aggrecasanase molecules, the DNA sequences obtained by those methods, and the protein encoded by those DNA sequences. This method entails utilizing the nucleotide sequence of the invention or portions thereof to design probes to screen libraries for the corresponding gene from other species or coding sequences or fragments thereof from using standard techniques. Thus, the present invention may include DNA sequences from other species, which are homologous to the human aggrecanase protein and can be obtained using the human sequence. The present invention may also include functional fragments of the aggrecanase protein, and DNA sequences encoding such functional fragments, as well as functional fragments of other related proteins. The a protein in the biological bility of such a fragment to function is determinable by assay of the assays described for the assay of the aggrecanase protein.

The aggrecanase proteins of the present invention may be produced by culturing a cell transformed with the DNA sequence of SEQ ID No. 2 ccomprising nucleootide #1 to #1045 or the nucleotide sequence comprising #1 to #1045 and the sequence comprising nucleotide #1 to #2217 of SEQ ID No. 4 and recovering and purifying aggrecanase protein from the culture medium. The purified expressed protein is substantially free from other proteinaceous materials with which it is co-produced, as well as from other contaminants. The recovered purified protein is contemplated to exhibit proteolytic aggrecanase activity cleaving aggrecan. Thus, the proteins of the invention may be further characterized by the ability to demonstrate aggrecan proteolytic activity in an asssay which determines the presence of an aggrecan-degrading molecule. These assays or the development thereof is within the knowledge of one skilled in the art. Such assays may involve contacting an aggrecan substrate with the aggrecanase molecule and monitoring the production of aggrecan fragments [see for example, Hughes et al., Biochem J 305: 799-804(1995); Mercuri et al, J. Bio Chem. 274:32387-32395 (1999)].

In another embodiment, the invention includes methods for developing inhibitors of aggrecanase and the inhibitors produced thereby. These inhibitors prevent cleavage of aggrecan. The method may entail the determination of binding sites based on the three dimnesional structure of aggrecanase and aggrecan and developing a molecule reactive with the binding site. Candidate molecules are assayed for inhibitory activity. Additional standard methods for developing inhibitors of the aggrecanse molecule are known to those skilled in the art. Assays for the inhibitors involve contacting a mixture of aggrecan and the inhibitor with an aggrecanase molecule followed by measurement of the aggrecanase inhibtion, for instance by detection and measurement of aggrecan fragments produced by cleavage at an aggrecanase susceptible site.

Another aspect of the invention therefore provides pharmaceutical compositions containing a therapeutically effective amount of aggrecanase inhibitors, in a pharmaceutically acceptable vehicle.

Aggrecanse-mediated degradation of aggrecan in cartilage has been implicated in osteoarthritis and other inflamatory diseases. Therefore, these compositions of the invention may be used in the treatment of diseases characterized by the degradation of aggrecan and/or an upregulation of aggrecanase. The compositions may be used in the treatment of these conditions or in the prevention thereof.

The invention includes methods for treating patients suffering from conditions characterized by a degradation of aggrecan or preventing such conditions. These methods, according to the invention, entail administering to a patient needing such treatment, an effective amount of a composition comprising an aggrecanase inhibitor which inhibits the proteilytic activity of aggrecanase enzymes.

Still a further aspect of the invention are DNA sequences coding for expression of an aggrecanase protein. Such sequences include the sequence of nucleotides in a 5′ to 3′ direction illustrated in FIG. 1 and DNA sequences which, but for the degeneracy of the genetic code, are identical to the DNA sequence of FIG. 1, and encode an aggrecanase protein. The invention further includes the nucleotide sequences set forth in SEQ ID Nos 2 and 3. Further included in the present invention are DNA sequences which hybridize under stringent conditions with the DNA sequence of FIG. 1or SEQ ID Nos 2 and 3 and encode a protein having the ability to cleave aggrecan. Preferred DNA sequences include those which hybridize under stringent conditions [see, T. Maniatis et al, Molecular Cloning (A Laboratory Manual), Cold Spring Harbor Laboratory (1982), pages 387 to 389]. It is generally preferred that such DNA sequences encode a polypeptide which is at least about 80% homologous, and more preferably at least about 90% homologous, to the sequence of set forth in SEQ ID No. 1. Finally, allelic or other variations of the sequences of FIG. 1 or SEQ ID No. 2 and 3, whether such nucleotide changes result in changes in the peptide sequence or not, but where the peptide sequence still has aggrecanase activity, are also included in the present invention. The present invention also includes fragments of the DNA sequence shown in FIG. 1 or SEQ ID Nos 2 and 3 which encode a polypeptide which retains the activity of aggrecanase.

The DNA sequences of the present invention are useful, for example, as probes for the detection of mRNA encoding aggrecanase in a given cell population. Thus, the present invention includes methods of detecting or diagnosing genetic disorders involving the aggrecanase, or disorders involving cellular, organ or tissue disorders in which aggrecanase is irregularly transcribed or expressed. The DNA sequences may also be useful for preparing vectors for gene therapy applications as described below.

A further aspect of the invention includes vectors comprising a DNA sequence as described above in operative association with an expression control sequence therefor. These vectors may be employed in a novel process for producing an aggrecanase protein of the invention in which a cell line transformed with a DNA sequence encoding an aggrecanase protein in operative association with an expression control sequence therefor, is cultured in a suitable culture medium and an aggrecanase protein is recovered and purified therefrom. This process may employ a number of known cells both prokaryotic and eukaryotic as host cells for expression of the polypeptide. The vectors may be used in gene therapy applications. In such use, the vectors may be transfected into the cells of a patient ex vivo, and the cells may be reintroduced into a patient. Alternatively, the vectors may be introduced into a patient in vivo through targeted transfection.

Still a further aspect of the invention are aggrecanase proteins or polypeptides. Such polypeptides are characterized by having an amino acid sequence including the sequence illustrated in SEQ ID No. 1, variants of the amino acid sequence of SEQ ID No. 1, including naturally occurring allelic variants, and other variants in which the protein retains the ability to cleave aggrecan characteristic of aggrecanase molecules. Preferred polypeptides include a polypeptide which is at least about 80% homologous, and more preferably at least about 90% homologous, to the amino acid sequence shown in SEQ ID No. 1. Finally, allelic or other variations of the sequences of SEQ ID No. 1, whether such amino acid changes are induced by mutagenesis, chemical alteration, or by alteration of DNA sequence used to produce the polypeptide, where the peptide sequence still has aggrecanase activity, are also included in the present invention. The present invention also includes fragments of the amino acid sequence of SEQ ID No. 1 which retain the activity of aggrecanase protein.

The purified proteins of the present inventions may be used to generate antibodies, either monoclonal or polyclonal, to aggrecanase and/or other aggrecanase-related proteins, using methods that are known in the art of antibody production. Thus, the present invention also includes antibodies to aggrecanase or other related proteins. The antibodies may be useful for detection and/or purification of aggrecanase or related proteins, or for inhibiting or preventing the effects of aggrecanase. The aggrecanase of the invention or portions thereof may be utilized to prepare antibodies that specifically bind to aggrecanase.

### DESCRIPTION OF THE DRAWINGS

FIG. 1 sets forth the nucleotide sequence of the isolated aggrecanase clone generated by consensus virtual sequence followed by the sequence of Hsa011374.

### DETAILED DESCRIPTION OF THE INVENTION

The human aggrecanase of the present invention comprises nucleotides #1 to #1045 of SEQ ID No. 2 or its complement set forth in SEQ ID no. 3. The human aggrecanase protein sequence comprises amino acids #1 to #242 set forth in SEQ ID No. 1. The full length sequence of the aggrecanase of the present invention is obtained using the sequences of SEQ ID No. 2 and 3 to design probes for screening for the full sequence using standard techniques.

The aggrecanase proteins of the present invention, include polypeptides comprising the amino acid sequence of SEQ ID No. 1 and having the ability to cleave aggrecan.

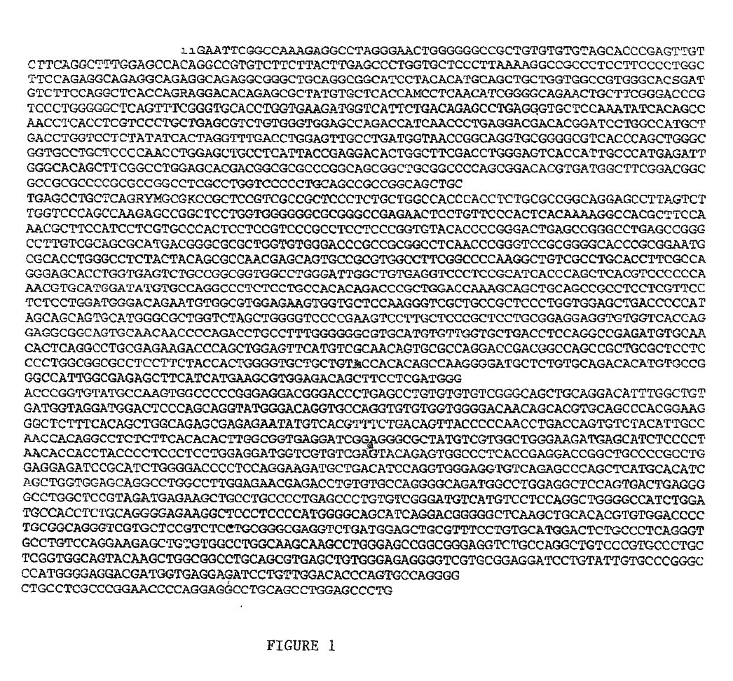

The aggrecanase proteins recovered from the culture medium are purified by isolating them from other proteinaceous materials from which they are co-produced and from other contaminants present. The isolated and purified proteins may be characterized by the ability to cleave aggrecan substrate. The aggrecanase proteins provided herein also include factors encoded by the sequences similar to those of FIG. 1 or SEQ ID Nos. 2 and 3, but into which modifications or deletions are naturally provided (e.g. allelic variations in the nucleotide sequence which may result in amino acid changes in the polypeptide) or deliberately engineered. For example, synthetic polypeptides may wholly or partially duplicate continuous sequences of the amino acid residues of SEQ ID NO. 1. These sequences, by virtue of sharing primary, secondary, or tertiary structural and conformational characteristics with aggrecanase molecules may possess biological properties in common therewith. It is know, for example that numerous conservative amino acid substitutions are possible without significantly modifying the structure and conformation of a protein, thus maintaining the biological properties as well. For example, it is recognized that conservative amino acid substitutions may be made among amino acids with basic side chains, such as lysine (Lys or K), arginine (Arg or R) and histidine (His or H); amino acids with acidic side chains, such as aspartic acid (Asp or D) and glutamic acid (Glu or E); amino acids with uncharged polar side chains, such as asparagine (Asn or N), glutamine (Gln or Q), serine (Ser or S), threonine (Thr or T), and tyrosine (Tyr or Y); and amino acids with nonpolar side chains, such as alanine (Ala or A), glycine (Gly or G), valine (Val or V), leucine (Leu or L), isoleucine (Ile or I), proline (Pro or P), phenylalanine (Phe or F), methionine (Met or M), tryptophan (Trp or W) and cysteine (Cys or C). Thus, these modifications and deletions of the native aggrecanase may be employed as biologically active substitutes for naturally-occurring aggrecanase and in the development of inhibitors other polypeptides in therapeutic processes. It can be readily determined whether a given variant of aggrecanase maintains the biological activity of aggrecanase by subjecting both aggrecanase and the variant of aggrecanase, as well as inhibitors thereof, to the assays described in the examples.

Other specific mutations of the sequences of aggrecanase proteins described herein involve modifications of glycosylation sites. These modifications may involve O-linked or N-linked glycosylation sites. For instance, the absence of glycosylation or only partial glycosylation results from amino acid substitution or deletion at asparagine-linked glycosylation recognition sites. The asparagine-linked glycosylation recognition sites comprise tripeptide sequences which are specifically recognized by appropriate cellular glycosylation enzymes. These tripeptide sequences are either asparagine-X-threonine or asparagine-X-serine, where X is usually any amino acid. A variety of amino acid substitutions or deletions at one or both of the first or third amino acid positions of a glycosylation recognition site (and/or amino acid deletion at the second position) results in non-glycosylation at the modified tripeptide sequence. Additionally, bacterial expression of aggrecanase-related protein will also result in production of a non-glycosylated protein, even if the glycosylation sites are left unmodified.

The present invention also encompasses the novel DNA sequences, free of association with DNA sequences encoding other proteinaceous materials, and coding for expression of aggrecanase proteins. These DNA sequences include those depicted in FIG. 1 in a 5′ to 3′ direction and those sequences which hybridize thereto under stringent hybridization washing conditions [for example, 0.1× SSC, 0.1% SDS at 65° C.; see, T. Maniatis et al, Molecular Cloning (A Laboratory Manual), Cold Spring Harbor Laboratory (1982), pages 387 to 389] and encode a protein having aggrecanase proteolytic activity. These DNA sequences also include those which comprise the DNA sequence of FIG. 1 and those which hybridize thereto under stringent hybridization conditions and encode a protein which maintain the other activities disclosed for aggrecanase.

Similarly, DNA sequences which code for aggrecanase proteins coded for by the sequences of FIG. 1 or SEQ ID NO. 2 or 3, or aggrecanase proteins which comprise the amino acid sequence of SEQ ID NO. 1, but which differ in codon sequence due to the degeneracies of the genetic code or allelic variations (naturally-occurring base changes in the species population which may or may not result in an amino acid change) also encode the novel factors described herein. Variations in the DNA sequences of FIG. 1 and SEQ ID NO. 2 and 3 which are caused by point mutations or by induced modifications (including insertion, deletion, and substitution) to enhance the activity, half-life or production of the polypeptides encoded are also encompassed in the invention.

Another aspect of the present invention provides a novel method for producing aggrecanase proteins. The method of the present invention involves culturing a suitable cell line, which has been transformed with a DNA sequence encoding a aggrecanase protein of the invention, under the control of known regulatory sequences. The transformed host cells are cultured and the aggrecanase proteins recovered and purified from the culture medium. The purified proteins are substantially free from other proteins with which they are co-produced as well as from other contaminants.

Suitable cells or cell lines may be mammalian cells, such as Chinese hamster ovary cells (CHO). The selection of suitable mammalian host cells and methods for transformation, culture, amplification, screening, product production and purification are known in the art. See, e.g., Gething and Sambrook, Nature, 293:620-625 (1981), or alternatively, Kaufman et al, Mol. Cell. Biol., 5(7):1750-1759 (1985) or Howley et al, U.S. Pat. No. 4,419,446. Another suitable mammalian cell line, which is described in the accompanying examples, is the monkey COS-1 cell line. The mammalian cell CV-1 may also be suitable.

Bacterial cells may also be suitable hosts. For example, the various strains of E. coli (e.g., HB101, MC1061) are well-known as host cells in the field of biotechnology. Various strains of B. subtilis, Pseudomonas, other bacilli and the like may also be employed in this method. For expression of the protein in bacterial cells, DNA encoding the propeptide of Aggrecanase is generally not necessary.

Many strains of yeast cells known to those skilled in the art may also be available as host cells for expression of the polypeptides of the present invention. Additionally, where desired, insect cells may be utilized as host cells in the method of the present invention. See, e.g. Miller et al, Genetic Engineering, 8:277-298 (Plenum Press 1986) and references cited therein.

Another aspect of the present invention provides vectors for use in the method of expression of these novel aggrecanase polypeptides. Preferably the vectors contain the full novel DNA sequences described above which encode the novel factors of the invention. Additionally, the vectors contain appropriate expression control sequences permitting expression of the aggrecanase protein sequences. Alternatively, vectors incorporating modified sequences as described above are also embodiments of the present invention. Additionally, the sequence of FIG. 1 or SEQ ID No. 2 and 3 or other sequences encoding aggrecanase proteins could be manipulated to express composite aggrecanase molecules. Thus, the present invention includes chimeric DNA molecules encoding an aggrecanase proteion comprising a fragment from FIG. 1 or SEQ ID No. 2 and 3 linked in correct reading frame to a DNA sequence encoding another aggrecanase polypeptide.

The vectors may be employed in the method of transforming cell lines and contain selected regulatory sequences in operative association with the DNA coding sequences of the invention which are capable of directing the replication and expression thereof in selected host cells. Regulatory sequences for such vectors are known to those skilled in the art and may be selected depending upon the host cells. Such selection is routine and does not form part of the present invention.

Various conditions such as osteoartritis are known to be characterized by degradation of aggrecan. Therfore, an aggrecanase protein of the present invention which cleaves aggrecan may be useful for the development of inhibitors of aggrecanase. The invention therefore provides compositions comprising an aggrecanase inhibitor. The inhibitors may be developed using the aggrecanase in screening assays involving a mixture of aggrecan substrate with the inhibitor followed by exposure to aggrecan. The compostions may be used in the treatment of osteoarthritis and other conditions exhibiting degradation of aggrecan. The invention further includes antibodies which can be used to detect aggrecanase and also may be used to inhibit the prooteolytic activity of aggrecanase.

The therapeutic methods of the invention includes administering the aggrecanase inhibitor compositions topically, systemically, or locally as an implant or device. The dosage regimen will be determined by the attending physician considering various factors which modify the action of the aggrecanase protein, the site of pathology, the severity of disease, the patient's age, sex, and diet, the severity of any inflamation, time of administration and other clinical factors. Generally, systemic or injectable administration will be initiated at a dose which is minimally effective, and the dose will be increased over a preselected time course until a positive effect is observed. Subsequently, incremental increases in dosage will be made limiting such incremental increases to such levels that produce a corresponding increase in effect, while taking into account any adverse affects that may appear. The addition of other known factors, to the final composition, may also effect the dosage.

Progress can be monitored by periodic assessment of disease progression. The progress can be monitored, for example, by x-rays, MRI or other imaging modalities, synovial fluid analysis, and/or clinical examination.

The following examples illustrate practice of the present invention in isolating and characterizing human aggrecanase and other aggrecanase-related proteins, obtaining the human proteins and expressing the proteins via recombinant techniques.

### EXAMPLES

### Example 1

Isolation of DNA

Potential novel aggrecanase family members were identified using a database screening approach. Aggrecanase-1 [Science284:1664-1666 (1999)] has at least six domains: signal, propeptide, catalytic domain, disintegrin, tsp and c-terminal. The catalytic domain contains a zinc binding signature region, TAAHELGHVKF and a “MET turn” which are responsible for protease activity. Substitutions within the zinc binding region in the number of the positions still allow protease activity, but the histidine (H) and glutamic acid (E) residues must be present. The thrombospondin domain of Aggrecanase-1 is also a critical domain for substrate recognition and cleavage. It is these two domains that determine our classification of a novel aggrecanase family member. The protein sequence of the Aggrecanase-1 DNA sequence was used to query against the GeneBank ESTs focusing on human ESTs using TBLASTN. The resulting sequences were the starting point in the effort to identify full length sequence for potential family members. The nucleotide sequence of the aggrecanase of the present invention is comprised of five EST's that contain homology over the catalytic domain and zinc binding motif of Aggrecanase-1.

This human aggrecanase sequence was isolated from a dT-primed cDNA library constructed in the plasmid vector pED6-dpc2(cite or description). cDNA was made from human stomach RNA purchased from Clontech. The probe to isolate the aggrecanase of the present invention was generated from the sequence obtained from the database search. The sequence of the probe was as follows: 5′-GTGAGGTTGGCTGTGATATTTGGAGCAC-3′. The DNA probe was radioactively labelled with ³²P and used to screen the human stomach dT-primed cDNA library, under high stringency hybridization/washing conditions, to identify clones containing sequences of the human candidate #5.

Fifty thousand library transformants were plated at a density of approximately 5000 transformants per plate on 10 plates. Nitrocellulose replicas of the transformed colonies were hybridized to the ³²P labeled DNA probe in standard hybridization buffer (1× Blotto[25× Blotto=%5 nonfat dried milk, 0.02% azide in dH2O]+1% NP-40+6× SSC+0.05% Pyrophosphate) under high stringency conditions (65° C. for 2 hours). After 2 hours hybridization, the radioactively labelled DNA probe containing hybridization solution was removed and the filters were washed under high stringency conditions (3× SSC, 0.05% Pyrophosphate for 5 minutes at RT; followed by 2.2× SSC, 0.05% Pyrophosphate for 15 minutes at RT; followed by 2.2× SSC, 0.05% Pyrophosphate for 1-2 minutes at 65° C. The filters were wrapped in Saran wrap and exposed to X-ray film for overnight. The autoradiographs were developed and positively hybridizing transformants of various signal intensities were identified. These positive clones were picked; grown for 12 hours in selective medium and plated at low density (approximately 100 colonies per plate). Nitrocellulose replicas of the colonies were hybridized to the ³²P labelled probe in standard hybridization buffer ((1× Blotto[25× Blotto=%5 nonfat dried milk, 0.02% azide in dH2O]+1% NP-40+6× SSC+0.05% Pyrophosphate) under high stringency conditions (65° C. for 2 hours). After 2 hours hybridization, the radioactively labelled DNA probe containing hybridization solution was removed and the filters were washed under high stringency conditions (3× SSC, 0.05% Pyrophosphate for 5 minutes at RT; followed by 2.2× SSC, 0.05% Pyrophosphate for 15 minutes at RT; followed by 2.2× SSC, 0.05% Pyrophosphate for 1-2 minutes at 65° C. The filters were wrapped in Saran wrap and exposed to X-ray film for overnight. The autoradiographs were developed and positively hybridizing transformants were identified. Bacterial stocks of purified hybridization positive clones were made and plasmid DNA was isolated. The sequence of the cDNA insert was determinedand is set forth in SEQ ID Nos. 2 and 3. This sequence has been deposited in the American Type Culture Collection 10801 University Blvd. Manassas, Va. 20110-2209 USA as PTA-2285. The cDNA insert contained the sequences of the DNA probe used in the hybridization.

The human candidate #5 sequence obtained aligns with several EST's in the public database, along with a human cDNA, hsa011374. Hsa011374 extends the aggrecanase sequence of the present invention about 2 kB at the 3′ end. When two gaps are inserted in the hsa0113745 sequence, the aggrecanase sequence of the present invention can be lined up to create a sequence that is about 40% homologous to Aggrecanase-1. The aggrecanase of the present invention contains the zinc biding region signature and a “MET turn”, however is missing the signal and propeptide regions. The hsa011374 extends our sequence to cover the disintegrin, tsp and c-terminal spacer. It is with these criteria that candidate #5 is considered a novel Aggrecanase family member.

The aggrecanse sequence of the invention can be used to design probes for further screening for full length clones containing the isolated sequence.

### Example 2

Expression of Aggrecanase

In order to produce murine, human or other mammalian aggrecanase-related proteins, the DNA encoding it is transferred into an appropriate expression vector and introduced into mammalian cells or other preferred eukaryotic or prokaryotic hosts including insect host cell culture systems by conventional genetic engineering techniques. Expression system for biologically active recombinant human aggrecanase is contemplated to be stably transformed mammalian cells, insect, yeast or bacterial cells.

One skilled in the art can construct mammalian expression vectors by employing the sequence of FIG. 1 or SEQ ID NO. 2 and 3, or other DNA sequences encoding aggrecanase-related proteins or other modified sequences and known vectors, such as pCD [Okayama et al., Mol. Cell Biol., 2:161-170 (1982)], pJL3, pJL4 [Gough et al., EMBO J., 4:645-653 (1985)] and pMT2 CXM.

The mammalian expression vector pMT2 CXM is a derivative of p91023(b) (Wong et al., Science 228:810-815, 1985) differing from the latter in that it contains the ampicillin resistance gene in place of the tetracycline resistance gene and further contains a XhoI site for insertion of cDNA clones. The functional elements of pMT2 CXM have been described (Kaufman, R. J., 1985, Proc. Natl. Acad. Sci. USA 82:689-693) and include the adenovirus VA genes, the SV40 origin of replication including the 72 bp enhancer, the adenovirus major late promoter including a 5′ splice site and the majority of the adenovirus tripartite leader sequence present on adenovirus late mRNAs, a 3′ splice acceptor site, a DHFR insert, the SV40 early polyadenylation site (SV40), and pBR322 sequences needed for propagation in E. coli.

Plasmid pMT2 CXM is obtained by EcoRI digestion of pMT2-VWF, which has been deposited with the American Type Culture Collection (ATCC), Rockville, Md. (USA) under accession number ATCC 67122. EcoRI digestion excises the cDNA insert present in pMT2-VWF, yielding pMT2 in linear form which can be ligated and used to transform E. coli HB 101 or DH-5 to ampicillin resistance. Plasmid pMT2 DNA can be prepared by conventional methods. pMT2 CXM is then constructed using loopout/in mutagenesis [Morinaga, et al., Biotechnology 84: 636 (1984). This removes bases 1075 to 1145 relative to the Hind III site near the SV40 origin of replication and enhancer sequences of pMT2. In addition it inserts the following sequence:

5′ PO-CATGGGCAGCTCGAG-3′

at nucleotide 1145. This sequence contains the recognition site for the restriction endonuclease Xho I. A derivative of pMT2CXM, termed pMT23, contains recognition sites for the restriction endonucleases PstI, Eco RI, SalI and XhoI. Plasmid pMT2 CXM and pMT23 DNA may be prepared by conventional methods.

pEMC2β1 derived from pMT21 may also be suitable in practice of the invention. pMT21 is derived from pMT2 which is derived from pMT2-VWF. As described above EcoRI digestion excises the cDNA insert present in pMT-VWF, yielding pMT2 in linear form which can be ligated and used to transform E. Coli HB 101 or DH-5 to ampicillin resistance. Plasmid pMT2 DNA can be prepared by conventional methods.

pMT21 is derived from pMT2 through the following two modifications. First, 76 bp of the 5′ untranslated region of the DHFR cDNA including a stretch of 19 G residues from G/C tailing for cDNA cloning is deleted. In this process, a XhoI site is inserted to obtain the following sequence immediately upstream from DHFR: 5′

CTGCAGGCGAGCCTGAATTCCTCGAGCCATCATG-3′
PstI Eco RI XhoI

Second, a unique ClaI site is introduced by digestion with EcoRV and XbaI, treatment with Klenow fragment of DNA polymerase I, and ligation to a ClaI linker (CATCGATG). This deletes a 250 bp segment from the adenovirus associated RNA (VAI) region but does not interfere with VAI RNA gene expression or function. pMT21 is digested with EcoRI and XhoI, and used to derive the vector pEMC2B1.

A portion of the EMCV leader is obtained from pMT2-ECAT1 [S. K. Jung, et al, J. Virol 63:1651-1660 (1989)] by digestion with Eco RI and PstI, resulting in a 2752 bp fragment. This fragment is digested with TaqI yielding an Eco RI-TaqI fragment of 508 bp which is purified by electrophoresis on low melting agarose gel. A 68 bp adapter and its complementary strand are synthesized with a 5′ TaqI protruding end and a 3′ XhoI protruding end which has the following sequence:

```
5′-CGAGGTTAAAAAACGTCTAGGCCCCCCGAACCACGGGGACGTGGTTTTCCTTT
    TaqI
GAAAAACACGATTGC-3′
                XhoI
```

This sequence matches the EMC virus leader sequence from nucleotide 763 to 827. It also changes the ATG at position 10 within the EMC virus leader to an ATT and is followed by a XhoI site. A three way ligation of the pMT21 Eco RI-16hoI fragment, the EMC virus EcoRI-TaqI fragment, and the 68 bp oligonucleotide adapter TaqI-16hoI adapter resulting in the vector pEMC2β1.

This vector contains the SV40 origin of replication and enhancer, the adenovirus major late promoter, a cDNA copy of the majority of the adenovirus tripartite leader sequence, a small hybrid intervening sequence, an SV40 polyadenylation signal and the adenovirus VA I gene, DHFR and β-lactamase markers and an EMC sequence, in appropriate relationships to direct the high level expression of the desired cDNA in mammalian cells.

The construction of vectors may involve modification of the aggrecanase-related DNA sequences. For instance, aggrecanase cDNA can be modified by removing the non-coding nucleotides on the 5′ and 3′ ends of the coding region. The deleted non-coding nucleotides may or may not be replaced by other sequences known to be beneficial for expression. These vectors are transformed into appropriate host cells for expression of aggrecanase-related proteins. Additionally, the sequence of FIG. 1 or SEQ ID No: 2 and 3 or other sequences encoding aggrecanase-related proteins can be manipulated to express a mature aggrecanase-related protein by deleting aggrecanase encoding propeptide sequences and replacing them with sequences encoding the complete propeptides of other aggrecanase proteins.

One skilled in the art can manipulate the sequences of FIG. 1 or SEQ ID No. 2 and 3 by eliminating or replacing the mammalian regulatory sequences flanking the coding sequence with bacterial sequences to create bacterial vectors for intracellular or extracellular expression by bacterial cells. For example, the coding sequences could be further manipulated (e.g. ligated to other known linkers or modified by deleting non-coding sequences therefrom or altering nucleotides therein by other known techniques). The modified aggrecanase-related coding sequence could then be inserted into a known bacterial vector using procedures such as described in T. Taniguchi et al., Proc. Natl Acad. Sci. USA, 77:5230-5233 (1980). This exemplary bacterial vector could then be transformed into bacterial host cells and a aggrecanase-related protein expressed thereby. For a strategy for producing extracellular expression of aggrecanase-related proteins in bacterial cells, see, e.g. European patent application EPA 177,343.

Similar manipulations can be performed for the construction of an insect vector [See, e.g. procedures described in published European patent application 155,476] for expression in insect cells. A yeast vector could also be constructed employing yeast regulatory sequences for intracellular or extracellular expression of the factors of the present invention by yeast cells. [See, e.g., procedures described in published PCT application WO86/00639 and European patent application EPA 123,289].

A method for producing high levels of a aggrecanase-related protein of the invention in mammalian, bacterial, yeast or insect host cell systems may involve the construction of cells containing multiple copies of the heterologous Aggrecanase-related gene. The heterologous gene is linked to an amplifiable marker, e.g. the dihydrofolate reductase (DHFR) gene for which cells containing increased gene copies can be selected for propagation in increasing concentrations of methotrexate (MTX) according to the procedures of Kaufman and Sharp, J. Mol. Biol., 159:601-629 (1982). This approach can be employed with a number of different cell types.

For example, a plasmid containing a DNA sequence for ann aggrecanase-related protein of the invention in operative association with other plasmid sequences enabling expression thereof and the DHFR expression plasmid pAdA26SV(A)3 [Kaufman and Sharp, Mol. Cell. Biol., 2:1304 (1982)] can be co-introduced into DHFR-deficient CHO cells, DUKX-BII, by various methods including calcium phosphate coprecipitation and transfection, electroporation or protoplast fusion. DHFR expressing transformants are selected for growth in alpha media with dialyzed fetal calf serum, and subsequently selected for amplification by growth in increasing concentrations of MTX (e.g. sequential steps in 0.02, 0.2, 1.0 and 5 uM MTX) as described in Kaufman et al., Mol Cell Biol., 5:1750 (1983). Transformants are cloned, and biologically active aggrecanase expression is monitored by the assays described above. Aggrecanase protein expression should increase with increasing levels of MTX resistance. Aggrecanase polypeptides are characterized using standard techniques known in the art such as pulse labeling with [35S] methionine or cysteine and polyacrylamide gel electrophoresis. Similar procedures can be followed to produce other related aggrecanase-related proteins.

As one example the aggrecanase gene of the present invention is cloned into the expression vector pED6 [Kaufman et al., Nucleic Acid Res. 19:44885-4490(1991)]. COS and CHO DUKX B11 cells are transiently transfected with the aggrecanase sequence of the invention (+/− co-transfection of PACE on a separate pED6 plasmid) by lipofection (LF2000, Invitrogen). Duplicate transfections are performed for each gene of interest: (a) one for harvesting conditioned media for activity assay and (b) one for 35-S-methionine/cysteine metabolic labeling.

On day one media is changed to DME (COS) or alpha (CHO) media+1% heat-inactivated fetal calf serum +/−100 μg/ml heparin on wells(a) to be harvested for activity assay. After 48 h (day 4), conditioned media is harvested for activity assay.

On day 3, the duplicate wells(b) were changed to MEM (methionine-free/cysteine free) media+1% heat-inactivated fetal calf serum+100 μg/ml heparin+100 μCi/ml 35S-methionine/cysteine (Redivue Pro mix, Amersham). Following 6 h incubation at 37° C., conditioned media was harvested and run on SDS-PAGE gels under reducing conditions. Proteins are visualized by autoradiography.

### Example 3

Biological Activity of Expressed Aggrecanase

To measure the biological activity of the expressed aggrecanase-related proteins obtained in Example 2 above, the proteins are recovered from the cell culture and purified by isolating the aggrecanase-related proteins from other proteinaceous materials with which they are co-produced as well as from other contaminants. The purified protein may be assayed in accordance with assays described above. Purification is carried out using standard techniques known to those skilled in the art.

Protein analysis is conducted using standard techniques such as SDS-PAGE acrylamide [Laemmli, Nature 227:680 (1970)] stained with silver [Oakley, et al. Anal. Biochem. 105:361 (1980)] and by immunoblot [Towbin, et al. Proc. Natl. Acad. Sci. USA 76:4350 (1979)].

The foregoing descriptions detail presently preferred embodiments of the present invention. Numerous modifications and variations in practice thereof are expected to occur to those skilled in the art upon consideration of these descriptions. Those modifications and variations are believed to be encompassed within the claims appended hereto.

What is claimed is:

1. An isolated DNA molecule comprising a DNA sequence set forth in SEQ ID NO. 2.

2. An isolated DNA molecule comprising a DNA sequence set forth in SEQ ID NO. 3.

3. An isolated DNA molecule comprising a DNA sequence set forth in SEQ ID NO. 4.

4. An isolated DNA molecule comprising a DNA sequence selected from the group consisting of
a) the sequence set forth in FIG. 1 or a fragment thereof;
b) the sequence of SEQ ID NO. 2,
c) the sequence of SEQ ID NO. 3
d) the sequence of SEQ ID NO. 3 from nucleotide #1 to #1045 and the sequence set forth in SEQ ID NO. 4 from nuclleotide #1 through 2217; and
(e) naturally occurring human allelic sequences and equivalent degenerative codon sequences of (a) through (d).

5. A vector comprising a DNA molecule of claim 1 in operative association with an expression control sequence therefor.

6. A host cell transformed with the DNA sequence of claim 1.

7. A host cell transformed with a DNA sequence of claim 2.

8. A method for producing a purified human aggrecanase protein, said method comprising the steps of:
(a) culturing a host cell transformed with a DNA molecule according to claim 1; and
(b) recovering and purifying said aggrecanase protein from the culture medium.

9. A method for producing a purified human aggrecanase protein, said method comprising the steps of:
(a) culturing a host cell transformed with a DNA molecule according to claim 2; and
(b) recovering and purifying said aggrecanase protein from the culture medium.

10. The method of claim 8, wherein said host cell is an insect cell.

11. A purified aggrecanase polypeptide comprising the amino acid sequence set forth in SEQ ID NO 1.

12. A purified aggrecanase polypeptide produced by the steps of
(a) culturing a cell transformed with a DNA molecule according to claim 3; and
(b) recovering and purifying from said culture medium a polypeptide comprising the amino acid sequence set forth in SEQ ID NO. 1.

13. An antibody that binds to a purified aggrecanase protein of claim 11.

14. A method for developing inhibitors of aggrecanase comprising the use of aggrecanase protein set forth in SEQ ID NO. 1 or a fragment thereof.

15. The method of claim 14 wherein said method comprises three dimensional structural analysis.

16. The method of claim 14 wherein said method comprises computer aided drug design.

17. A composition for inhibiting the proteolytic activity of aggrecanase comprising a peptide molecule which binds to the aggrecanase inhibiting the proteolytic degradation of aggrecane.

18. A method for inhibiting the cleavage of aggrecan in a mammal comprising administering to said mammal an effective amount of a compound that inhibits aggrecanase activity.

19. The sequence of Hsa011374 SEQ ID NO. 4 and the protein sequences encoded thereby for use in developing aggrecanase inhibitory compounds.


SEQUENCE LISTING (6 sequences)

SEQ ID NO: 1 - protein, 242 aa
HPSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLR

SEQ ID NO: 2 - DNA (genomic), 1045 bp; unknown-stranded, unknown
gaattcggccaaagaggcctacgagtgtggtcaggatggagaggtaggacaggaaggagggctgaatgcggagtggggacggacgtccggagggctggctggaagctcgcgcgcccctcccacggggcgggcgctacctgagcaggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcagggacgaggtgaggttggctgtgatatttggagcaccctcaggctctgtcagaatgaccatcttcaccaggtgcacccgaaactgagcccccagggacgggtcccgaagcagttctgccccgatgttgaggttggtgagcacatagcgctctgtgtcctcctggtgagcctggaagacatcggggcccacggccaccagcagctccaggtgtaggatgccgcctgcagcccgcctctgcctctgcctctgcctctggaagccaggggaaggagggcggccttttaagggagcaccagggctcaagtaagaagacacggcctgtggctccaaagcctgaagacaactcgggtgctacacacacagcggccccccagttcccttccggcgttcgcatctctcatccccatcccggatcttggggaggtcctcggcttgccccagtcaaactcgaggttctccctatagtgagtcgtattaatttcagaggagtatttagaagagaagctgaagctgtcgagacaaacgaaactagtg

SEQ ID NO: 3 - DNA (genomic), 1045 bp; unknown-stranded, unknown
cactagtttcgtttgtctcgacagcttcagcttctcttctaaatactcctctgaaattaatacgactcactatagggagaacctcgagtttgactggggcaagccgaggacctccccaagatccgggatggggatgagagatgcgaacgccggaagggaactggggggccgctgtgtgtgtagcacccgagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcaggtagcgcccgccccgtgggaggggcgcgcgagcttccagccagccctccggacgtccgtccccactccgcattcagccctccttcctgtcctacctctccatcctgaccacactcgtaggcctctttggccgaattc

SEQ ID NO: 4 - DNA (genomic), 2217 bp; unknown-stranded, unknown
cagcttcggcctggagcacgacggcgcgcccggcagcggctgcggccccagcggacacgtgatggcttcggaacggcgccgccccgcgccggcctcgcctggtccccctgcagccgccggcagctgctgagcctgctcagacccgtccctccgtcgccgctccctctgctggccacccacctctgcgccggcaggagccttagtcttggtcccagccaagagccggctcctggtggggggcgcgggccgagaactcctgttcccactcacaaaaggccacgcttccaaacgcttccatcctcgtgcccactcctccgtcccgcctcctcccggtgtacaccccgggactgagccgggcctgagccgggccttgtcgcagcgcatgacgggcgcgctggtgtgggacccgccgcggcctcaacccgggtccgcggggcacccgcggaatgcgcacctgggcctctactacagcgccaacgagcagtgccgcgtggccttcggccccaaggctgtcgcctgcaccttcgccagggagcacctggtgagtctgccggcggtggcctgggattggctgtgaggtccctccgcatcacccagctcacgtccccccaaacgtgcatggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctg

SEQ ID NO: 5 - protein, 365 aa
MDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVGGVRAQLMHISWWSRPGLGERDLCARGRWPGGSSDX

SEQ ID NO: 6 - protein, 738 aa
SFGLEHDGAPGSGCGPSGHVMASERRRPAPASPGPPAAAGSCXACSDPSLRRRSLCWPPTSAPAGALVLVPAKSRLLVGGAGRELLFPLTKGHASKRFHPRAHSSVPPPPGVHPGTEPGLSRALSQRMTGALVWDPPRPQPGSAGHPRNAHLGLYYSAAEQCRVAFGPKAVACTFAREHLVSLPAVAWDWLXGPSASPSSRPPKRAWICARPSPATQTRWTKAAAAASSFLSWMGQNVAWRSGAPRVAAAPWWSXPPXQQCMGAGLAGVPEVLAPAPAEEVWSPGGGSATTPDLPLGGVHVLVLTSRPRCATLRPARRPSWSSCRNSAPGPTASRCAPPLAAPPSTTGVLLYHTAKGMLCADTCAGPLARASSXSVETASSMGPGVCQVAPGRTGPXACVCRAAAGHLAVMVGWTPSRYGTGARCVVGTTARAAHGRALSQLAERENMSRFXQLPPTXPVSTLPTTGLSSHTWRXGSEGAMSWLGRXASPLTPPTPPSWRMVVSSTEWPSPRTGCPAWRRSASGDPSRKMLTSRWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEP